(19) United States
(12) Patent Application Publication
(10) Pub. No.: US 2021/0059763 A1
(43) Pub. Date: Mar. 4, 2021

(54) System for Intracranial Imaging and Treatment

(71) Applicant: ArcheOptix Biomedical Inc., Kingston (CA)

(72) Inventors: Jason David Richard Riley, Ottawa (CA); Douglas James Cook, Odessa (CA)

(21) Appl. No.: 16/977,163

(22) PCT Filed: Mar. 1, 2019
(86) PCT No.: PCT/CA2019/050243

§ 371 Date: Sep. 1, 2020

Related U.S. Application Data
(60) Provisional application No. 62/636,921, filed on Mar. 1, 2018.

(51) Int. Cl. A61B 34/20; A61B 1/313; A61B 1/06; A61B 1/00; A61B 1/018; A61M 3/02; A61B 18/08; A61N 1/05

(52) U.S. Cl. CPC A61B 34/20; A61B 1/313; A61B 1/0684; A61B 1/00087; A61B 2018/00595; A61M 3/02; A61B 18/082; A61N 1/0529; A61B 1/018

(57) ABSTRACT

The present invention provides a system for intracranial imaging and treatment of an intracranial region including: (a) a catheter probe suitable for insertion into the intracranial region, the catheter probe including: (i) a catheter housing; (ii) an optical probe including one or more optical emitters; and (iii) an optional surgical tool, with the optical probe and the surgical tool located within the housing; and (b) an imaging plate configured for fixed attachment through a plurality of fixed attachment points to a surface of the intracranial region being imaged and treated. The imaging plate includes an array of sensors, each sensor including an optical receiver. The optical emitters are configured to emit light in proximity to the intracranial region being imaged. The array of sensors is configured to measure transmitted light to determine the status of the intracranial region being imaged.

### CROSS-REFERENCE TO RELATED APPLICATIONS

This application is the United States national phase of International Application No. PCT/CA2019/050243 filed Mar. 1, 2019, and claims priority to U.S. Provisional Patent Application No. 62/636,921 filed Mar. 1, 2018, the disclosures of which are hereby incorporated by reference in their entirety.

### BACKGROUND OF THE INVENTION

### Field of the Invention

The present invention pertains to the field of optical imaging and in particular to devices for optical imaging to detect hematoma.

### Description of Related Art

The major cause of death in traumatic brain injury (TBI) is bleeding in and around the brain.

Of particular concern are acute bleeds such as epidural, subdural and intracerebral hematomas that can cause direct brain compression and/or brain swelling and can lead to permanent brain damage and death.

These injuries may be missed in early detection because early clinical signs may be occult or limited in severity. Subsequent clinical deterioration occurs rapidly at variable times from the original injury. Early detection of lesions, prior to clinical decline, can facilitate rapid surgical intervention and improve outcomes.

The clinical need is to be able to reduce pressure on the brain by removing intracranial hematomas in an effective and minimally damaging way. To perform this task there is a need to target a surgical approach to precisely guide a surgeon to an intracerebral hematoma with the least damaging, minimally invasive, approach possible. Furthermore, there is a need to determine the extent of hematoma evacuation to ensure a maximal resection of the hematoma. Finally, the risk of immediate re-accumulation of hematoma following surgery may be avoided by intraoperative imaging of the hematoma following the initial resection to observe for additional bleeding in the interval following resection. In summary, there is a need for a targeted approach to intracerebral hematoma evacuation that decreases the risk of hematoma re-accumulation in the immediate postoperative phase to improve patients outcomes and decrease need for repeat procedures.

Currently head injuries are typically evacuated by one of two means. If the bleed is superficial (based on CT imaging), a hole is drilled and the blood is drained using suction and irrigation or, in some cases, blood is allowed to drain spontaneously into a drainage tube. In the case of deeper or more extensive bleeding a larger opening in the skull called a craniotomy is required to expose the region of bleeding and enter the brain to evacuate the clot completely. Craniotomy is higher risk, associated with worse outcomes and requires greater recovery than a burrhole.

To evacuate a deep and/or large intracerebral hematoma, a catheter may be inserted into the intracranial space through a single burr hole and into the brain directly to reach the hematoma. Suction can be applied to the catheter, irrigation delivered through the catheter, and physical disruption by moving the catheter in and out of the centre of the hematoma are used to extract blood from the intracranial space. The objective of such techniques is to guide the catheter/syringe through the bleed and extract all the blood. Using existing technologies, this approach can only be achieved using a priori images.

Problems with such an approach include:
a. Hematoma may be missed due to inaccurate navigation techniques;
b. The bleed may evolve between imaging and evacuation and the extent of resection may be underestimated, leaving residual hematoma;
c. As the hematoma is evacuated the location of remaining hematoma may not be clear due to shift of the brain or the hematoma, negating the navigation based on the a priori image;
d. After evacuation of the hematoma there may be occult re-accumulation of the hematoma in the interval immediately following surgery. This is not realized due to a lack of real time imaging to follow intraoperatively and post-operatively.

Although prior art systems employing Non-invasive Diffuse Optical Imaging (DOI), be it near-infrared spectroscopy (NIRS) or diffuse optical tomography (DOT), possess certain advantages over other imaging modalities in that they are “non-invasive” or “minimally-invasive” (in the case of fluorescent systems where a bio-marking tag is injected into the blood), in such systems, imaging of the bleed event is typically conducted with all sensors (emitters and detectors) on the outside of the head, thus relying on reflectance geometry. A typical ‘best estimate’ of imaging depth for such “external” NIR systems is about 3.5 cm into tissue in a reflectance geometry.

In the case of small animal imaging, transmission geometries may be employed, but only where the animal is so small that a reasonable amount of NIR light can be detected in the transmission geometry. As such, while the use of transmission geometries is known, it has been precluded from most human based imaging systems based on the simple fact that human bodies are too large to permit sufficient NIR energy to be transmitted without creating risk to the tissue.

Accordingly, there exists a need for systems and methods that allow for the imaging and monitoring of a bleed event concurrently with its evacuation/treatment, and wherein the bleed event may be located at a depth that cannot be imaged using prior art systems and methods.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a system for intracranial imaging and treatment. In accordance with an aspect of the present invention, there is provided a system for intracranial imaging and treatment of an intracranial region, the system comprising: (a) a catheter probe suitable for insertion into the intracranial region, the catheter probe comprising: (i) a catheter housing; (ii) an optical probe comprising one or more optical emitters; and (iii) an optional surgical tool; wherein the optical probe and the surgical tool are located within the housing; and (b) an imaging plate configured for fixed attachment through a plurality of attachment points to a surface of the intracranial region being imaged and treated, the imaging plate comprising an array of sensors, each sensor comprising an optical receiver; wherein the one or more optical emitters is configured to emit light in proximity to the region being imaged, and the array of sensors is configured to measure transmitted light to determine the status of the region being imaged. In embodiments wherein the region being imaged includes a bleed event, the surgical tool is configured to treat the bleed event.

In accordance with another aspect of the present invention, there is provided a method for imaging and treatment of an intracranial region of a subject, comprising the steps of: providing a system comprising: (a) a catheter probe suitable for insertion into the intracranial region, the catheter probe comprising: (i) a catheter housing; and (ii) an optical probe comprising one or more optical emitters, wherein the optical probe is located within the housing; (b) an imaging plate configured for fixed attachment through a plurality of attachment points to a surface of the intracranial region being imaged and treated, the imaging plate comprising an array of sensors, each sensor comprising an optical receiver; wherein the one or more optical emitters is configured to emit light in proximity to the region being imaged, and the array of sensors is configured to measure transmitted light to determine the status of the region being imaged; attaching the imaging plate through a plurality of attachment points to a surface of the intracranial region of the subject; inserting the catheter probe into the intracranial region of the subject; obtaining an image of the intracranial region by interrogating the intracranial region with light emitted by the one or more optical emitters and detecting transmitted light with the sensors located on the imaging plate. In embodiments wherein the system comprises a surgical tool located within the housing, the method further comprises the step of deploying the surgical tool to treat the intracranial region.

In accordance with another aspect of the present invention, there is provided a system for intracranial imaging and treatment of an intracranial region, the system comprising: (a) an imaging subsystem comprising (i) an optical probe comprising one or more optical emitters located within a catheter housing; and (ii) an imaging plate configured for fixed attachment through a plurality of fixed attachment points to a surface of the intracranial region being imaged and treated, the imaging plate comprising an array of sensors, each sensor comprising an optical receiver; wherein the one or more optical emitters is configured to emit light in proximity to the region being imaged, and the array of sensors is configured to measure transmitted light to determine the status of the region being imaged; and (b) an optional treatment subsystem comprising: (i) a surgical tool located within the catheter housing. In embodiments wherein the region being imaged includes a bleed event, the surgical tool is configured to treat the bleed event.

### BRIEF DESCRIPTION OF THE DRAWINGS

FIG. 1 is a schematic depiction of an imaging plate in accordance with one embodiment of the invention.

FIG. 2 is a schematic depiction of a catheter probe in accordance with one embodiment of the invention.

FIG. 3 is a schematic depiction of a single sensor in the imaging array in accordance with one embodiment of the invention.

FIG. 4 is a schematic depiction of a mechanical arm in accordance with one embodiment of the invention.

FIG. 5 is a schematic depiction of a flexible imaging plate in accordance with one embodiment of the invention.

FIG. 6 is a schematic depiction of the relative arrangement of components of an imaging/treatment system in accordance with one embodiment of the invention.

FIG. 7 is a schematic depiction of an alternative sensor in accordance with one embodiment of the invention.

FIGS. 8, 9A and 9B depict one configuration of the device suitable for placement over the site of injury in accordance with one embodiment of the invention.

FIGS. 10A and 10B depict another configuration of the device suitable for placement over the site of injury in accordance with one embodiment of the invention.

FIG. 11 is a schematic depiction of one embodiment employing a fixed plate in combination with a flexible imaging plate.

### DESCRIPTION OF THE INVENTION

As used herein, the term “about” refers to a +/−10% variation from the nominal value. It is to be understood that such a variation is always included in a given value provided herein, whether or not it is specifically referred to.

Unless defined otherwise, all technical and scientific terms used herein have the same meaning as commonly understood by one of ordinary skill in the art to which this invention belongs.

The present invention provides a system for imaging and monitoring of an intracranial region concurrent with treatment of the region being monitored.

To avoid the limitations in imaging depth achievable with externally located imaging systems, the present invention provides an imaging system that employs transmission geometries for the imaging of large (e.g., human) bodies through the use of an optical probe that can be deployed inside the body, for example, by locating the emitting devices in an intraoperative (catheter) probe. Using such an intraoperative probe affords the potential to increase the imaging depth to at least the depth to which the probe can be safely inserted (e.g., up to about 7 cm). Such an exemplary upper limit is calculated based on surgical risk factors of probe placement, and the ability to send and receive photons through tissue within the limitations of sensor technology and allowable source intensity (i.e., without causing damage to tissue). By placing the probe in the intracranial space and/or brain, it is possible to achieve greater resolution and improved depth penetration.

This unique approach also allows rapid imaging by incorporating motion (i.e., positional comparison) as part of the processing. Generally, in NIRS, a moving sensor creates noise, because NIRS inherently relies on geometry to derive useful information (particularly where that signal level is small), and because the mathematical models used typically do not respect an evolving geometry. The use of the motion itself as signal by using a differential measure over space was previously demonstrated in PCT Publication No. WO 2015/070348, the disclosure of which is incorporated herein by reference. In a preferred embodiment, the present system combines the use of motion with a transmission geometry.

The system in accordance with the present invention can also employ a temporal comparison to create images by going back over the region being imaged in a known and controlled manner. By incorporating aspects of both traditional static imaging and the motion as signal model, an approach has been developed that allows the initial creation of an image of the state of the bleed at the start of a surgery and then re-evaluation of the state of the bleed as treatment progresses.

The system is made possible by the use of an electro-mechanically monitored sensor (imaging) plate. Combination of the imaging plate with the controlled intraoperative placement of the optical probe (in which the coordinate system geometry of the probe is constrained to the same coordinate system as the sensor plate thus obviating the need for registration), allows the precise control needed to allow a differential algorithm based on a moving sensor to work.

While real-time absolute imaging in NIR is the goal, due to the subtleties of heterogeneous tissue and their effect on the data, this has previously been a very difficult task to achieve. The present invention therefore provides a real-time approach which can create an image that provides absolute information about the status of the intracranial region being interrogated. For example, when used to monitor a bleed event, the present system can be used to obtain absolute information about the presence or absence of a blood volume, which can be continuously compared to a priori knowledge obtained with respect to the initial state of the bleed event.

In one embodiment, the present invention therefore also provides a system that can monitor changes in or evolution of the bleed event during treatment. In such an embodiment, the system of the present invention can thus confirm the complete evacuation/treatment of the bleed event, avoiding residual injury.

The present invention also provides a system that can be used to continuously monitor a surgical site for further bleed events during post-operative recovery. The use of NIR allows continuous monitoring without exposing the patient to unsafe amounts of irradiating energy that are characteristic of imaging methods such as CT imaging. In addition, since the present invention contemplates the use of an imaging plate that can be securely affixed to the skull, the catheter probe with the NIR source can be left in place during post-operative monitoring, thus minimizing surgical prep time in the event that a further bleed event is detected.

In accordance with the present invention, the system for intracranial imaging comprises an optical probe comprising one or more optical emitters for illuminating the region being interrogated. In accordance with one embodiment, the optical probe is located within a catheter probe that is configured for insertion into the intracranial region being interrogated. In a preferred embodiment, the optical emitters emit NIR light. The light is transmitted through the tissue being interrogated and the transmitted light is detected by an array of sensors located on an imaging plate. In a preferred embodiment, the tissue being interrogated includes a bleed event.

In one embodiment, the method for imaging and treatment of an intracranial region of a subject comprises the step of obtaining an image of the intracranial region by interrogating the intracranial region with light emitted by the one or more optical emitters and detecting transmitted light with the sensors located on the imaging plate.

The system therefore also comprises an imaging plate comprising an array of sensors, each sensor comprising an optical receiver. In those embodiments in which the system is employed to locate and image a bleed event, the optical emitters emit light in proximity to the bleed event, and the array of sensors measure transmitted light to determine in real-time the location and status of the bleed event.

The sensor array located in the imaging plate may be arranged in any suitable configuration, including but not limited to a grid-like “N×M” configuration, a radially disposed “starburst” configuration, or in a series of concentric circles.

The imaging plate can be formed of a rigid or flexible material. The use of flexible materials allows the plate to conform to the body part being imaged, thereby ensuring optimum contact between the receiver and the surface being imaged. The use of rigid material to form the imaging plate may be suitable in cases where the imaging plate can be preformed to conform to the shape of the body part being imaged while in a deformable state, and subsequently hardened to assume the rigid form.

In one embodiment, 3D printing technologies are used to manufacture a custom fitted imaging plate, the shape of the plate being based on a priori images of the patient's head obtained using, for example, CT or MRI imaging processes. The use of customized imaging plates can minimize (or even obviate) the need for sensors having shape recovery capabilities.

In the case of both flexible and rigid imaging plates, the sensors employed in the imaging array are typically independently displaceable to ensure that each receiver can maintain optimum contact with the uneven surface being imaged.

In accordance with one embodiment, each sensor further comprises a biasing mechanism to ensure optimum contact with the surface is maintained, including but not limited to spring-like mechanisms or the use of suitable resilient materials that hold the receiver in the optimum contact position.

In one embodiment, each sensor includes a fixed housing having located within it a plunger configured to engage the receiver. In a further embodiment, each sensor further comprises a positional sensor to measure displacement.

In one embodiment, each sensor comprises a linear displacement sensor (LDS) provided to determine the relative radial position of each sensor. Measurement of the radial position combined with knowledge of the physical x-y geometry of the array allows for the determination of the underlying shape of the head and the exact position of each sensor on that surface.

In one embodiment, the sensor is an LDP (linear displacement potentiometer), comprising a variable resistor inside a fixed housing, the variable resistor changing the resistance of the LDP to give the ‘signal’ that describes the linear displacement. In one such embodiment, the resistor is hollow and contains an optical fiber, such that the tip of the fiber is always in contact with the surface, and its linear displacement is always known. The optical fiber also acts as an optical sensor, relaying the photons detected at the surface back to a remote sensor or, in an alternative embodiment, deliver photons from a remote light source.

In one embodiment, the sensor used in the present system is a ‘LDP-Photonic Sensor’, which employs optic fibers as the “barrel” of the LDP's, thus combining the LDP variable resistor and optical sensor as a single component, thus reducing the footprint of the sensor.

Such optical sensors could be configured as single channel, or as multi channel (by use of bi/tri-furcated fibers/one way mirrors, optic filters etc). This would allow a unit to be both a source and/or detector and also to function at multiple wavelengths.

In one embodiment, the imaging plate is located on a helmet scaffold. The helmet scaffold is shaped to fit the patient's head and is provided with a high density of openings, each of which may be adapted to house a sensor and/or receive a catheter.

In one embodiment, such a helmet could be designed to exhibit contrast in an a priori imaging system (e.g. CT/MRI), which may be used to localise the optimum positioning of sensors to treat/monitor the bleeding event. This embodiment allows the measurements to be taken in the reference frame of the original image, thus requiring no registration is required.

In one embodiment, the imaging plate is configured to be fixedly attached to the surface of the intracranial region being imaged. Any suitable attachment mechanism can be used, including, but not limited to, surgical screws that can be temporarily inserted into the skull, or the use of biologically compatible adhesives for attaching the plate to the skin of the subject undergoing treatment.

In one embodiment, the method for imaging and treatment of an intracranial region of a subject comprises the step of attaching the imaging plate through a plurality of attachment points to a surface of the intracranial region of the subject.

In one embodiment, the system also comprises a mechanical arm system comprising one or more mechanical arms, which are provided to control the relative positions of the components of the system. For example, the relative positions of the catheter probe and the imaging plate are controlled through the use of a mechanical arm linking these two components. The mechanical arm is connected to the respective components via a mechanical arm housing.

By controlling and monitoring the positions of the catheter probe relative to the imaging plate, geometric control of the imaging system can be achieved.

In one embodiment, an optional mechanical arm is provided to assist with supporting the weight of all components in the system.

In one embodiment, the method for imaging and treatment of an intracranial region of a subject comprises the step of inserting the catheter probe into the intracranial region of the subject.

In one embodiment of the device, the imaging plate is placed over the site of intracranial region being imaged and the catheter probe accesses the site of the injury from the side of the imaging plate through a burr hole formed in the skull.

In one embodiment of the device, the imaging plate is placed over the site of intracranial region being imaged and the catheter probe accesses the site of the intracranial region being imaged through an opening in the middle of the imaging plate.

In accordance with a preferred embodiment of the present invention, a surgical tool is provided within the catheter probe housing. In a further embodiment, the surgical tool is configured to treat the bleed event. In a preferred embodiment, the surgical tool is a syringe provided to aspirate the blood from the intracranial region.

It is also contemplated that the present system of intracranial imaging can be used in conjunction with other treatment processes. In such an alternative configuration, the system can be deployed with any surgical tool that can fit within the catheter probe housing, including but not limited to: a syringe, a cauterization probe to cauterize bleeding blood vessels, a irrigation jet for irrigation of tissue, or an electrode stimulator for stimulating brain tissue.

In one embodiment, the catheter probe further comprises a position control mechanism for controlling the position of the optical probe relative to the surgical tool within the catheter housing.

In an alternative embodiment, the present invention may be conceived as a system comprising an imaging subsystem configured to image the tissue being interrogated, and an optional treatment subsystem configured to treat the tissue. In this embodiment, the imaging subsystem comprises an optical probe comprising one or more optical emitters located within a catheter housing; and an imaging plate comprising an array of sensors, each sensor comprising an optical receiver. The imaging plate is for attachment to a surface of the intracranial region being imaged through a plurality of attachment points. In one embodiment, the treatment subsystem comprises a surgical tool located within the catheter housing. In a preferred embodiment, the surgical tool is configured to treat the bleed event.

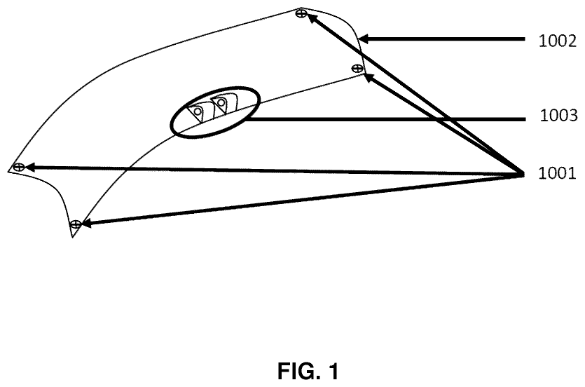

FIG. 1 is a schematic depiction of an imaging plate (1002) and the fixed attachment points (1001) used to anchor it to the head (via screws or other attachment mechanism). Also shown is a mechanical arm housing (1003) for attachment of a mechanical arm connection to a catheter probe (not shown), which provides geometrical control of the catheter probe. Also not shown are the sensors which are arrayed on the imaging plate.

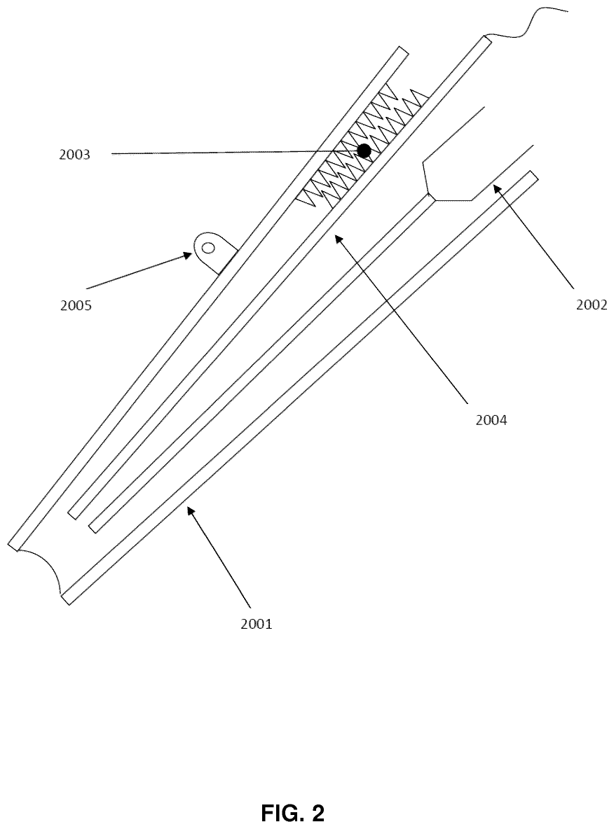

FIG. 2 is a schematic depiction of a catheter probe comprising a surgical catheter housing (2001) having located therein a syringe or similar surgical tool (2002) for removing the bleed and an LED probe (2004). The position of the LED probe relative to the catheter housing is controlled by a position control mechanism (2003). The position of the catheter relative to the plate is controlled by a mechanical arm (not shown) attached at the mechanical arm housing (2005).

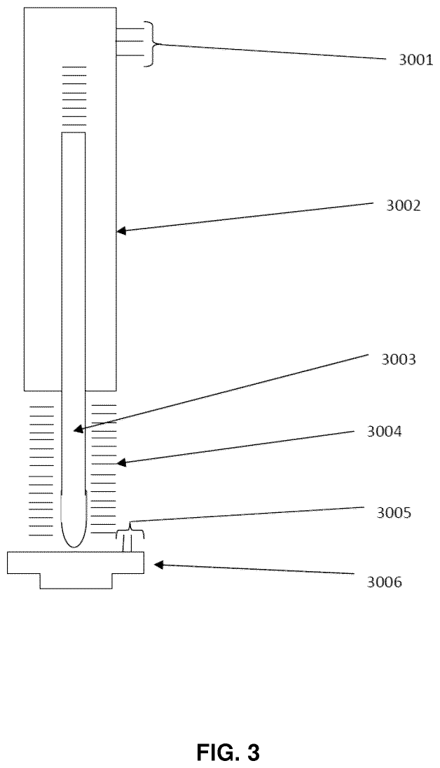

FIG. 3 is a schematic depiction of a single sensor in the imaging array, in accordance with one embodiment. In order to conform to the head, each sensor (receiver (3006)) is attached to a positional sensor for displacement as well as having its own sensor connections (3005) to the main imager. In the embodiment depicted in FIG. 3, a linear displacement sensor (LDS) with connectors (3001), a fixed housing (3002) and a plunger (3003) are shown. In this embodiment, a spring arrangement (3004) is positioned around the plunger to mechanically stabilise the sensor. When deployed, the spring ensures the receiver 3006 is firmly stabilized in place on the surface being imaged.

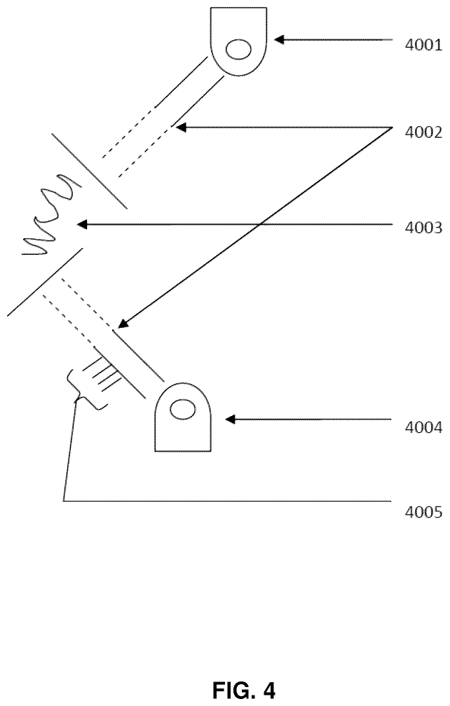

FIG. 4 is a schematic depiction of a mechanical arm (4003) having two ends (4002) extending between a housing (4001) on the catheter probe and a housing (4004) on the imaging plate. The arm (4003) is a robotic arm allowing for a full range of motion between the plate and catheter as appropriate to the surgery and imaging, and is connected to the main imager via a relational positional control i/o connection (4005).

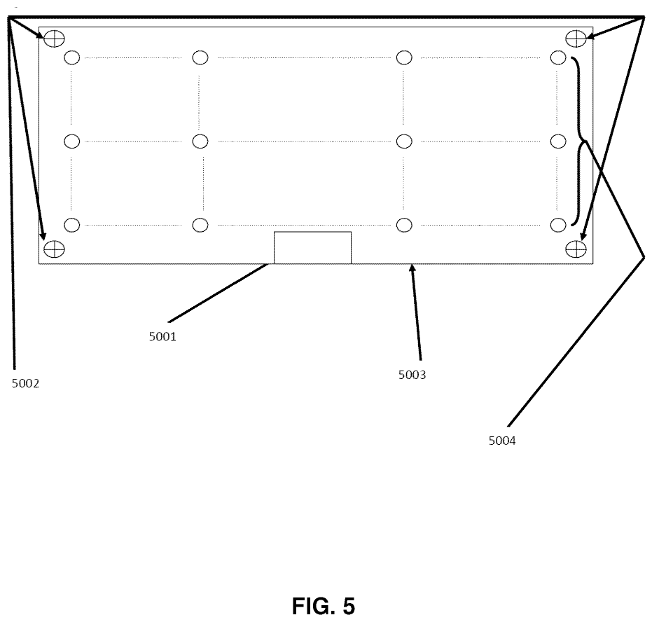

FIG. 5 is a schematic depiction of additional details of a flexible imaging plate in accordance with one embodiment. Shown is a mechanical arm housing (5001), flexible imaging plate (5003), and multiple fixed attachment points (5002). Also depicted is an array of sensors (5004). Although this array has been given dimensions N×M=3×4, this is not intended to be limiting, and any size and shape of sensor array may be used.

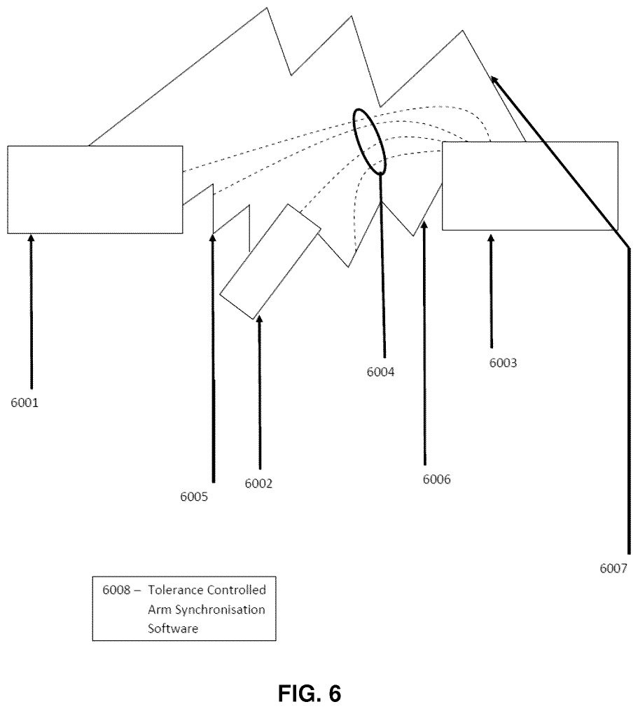

FIG. 6 is a schematic depiction of the relative arrangement of components of an imaging/treatment system in accordance with one embodiment, including a catheter probe (6002), an imaging plate (6001), and a mechanical arm (6005) connecting the imaging plate to the catheter probe. Also depicted are surgical robotic control unit (6003) and its catheter control arm (6006). Further added features are sensor controls (6004) for the imager which input data from the imaging system components to the surgical control unit (6003). Also depicted is an optional control arm (6007) which may be provided to assist with supporting the weight of all the components. An overall control system (6008) is provided to handle integration with tolerance control added to each arm.

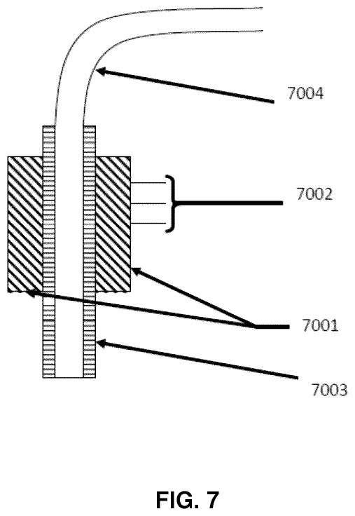

FIG. 7 depicts an optional variant of ‘LDP-Photonic Sensor’ that can be used in the present imaging/treatment system. This embodiment includes a standard LDP housing (7001) and 3-point LDP connector (7002). Also depicted is an optic fiber end housing (7003) acting as the LDP barrel and an optic fiber (7004) leading to the photo-detector.

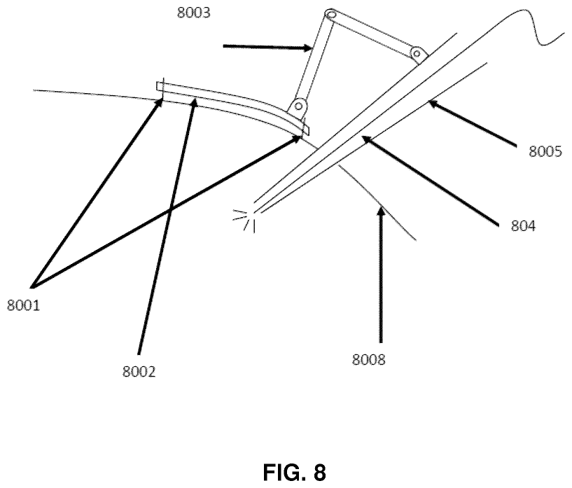

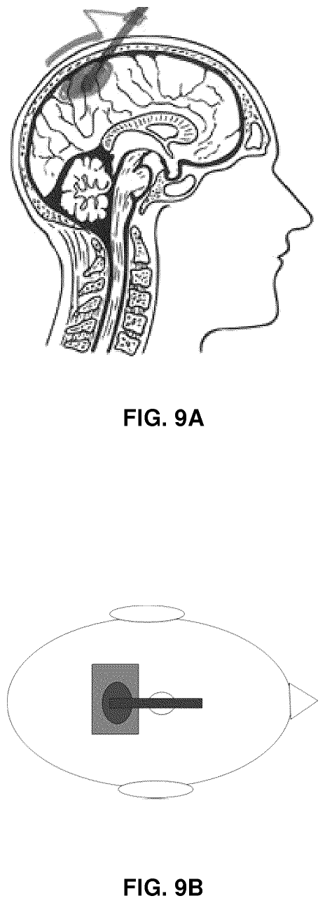

FIGS. 8, 9A and 9B describe embodiments of the device where the imaging plate is placed over the injury and the catheter accesses the site of the injury from the side of the imaging plate through a burr hole formed in the skull. FIG. 8 depicts the relative arrangement of the components of the imaging system in accordance with this embodiment. Imaging plate (8002) is provided as an array of NIR sensors fixed to the surface of the skull (8008) via mechanical attachments (8001). Control arm (8003) is provided to connect catheter probe (8005) to the imaging plate (8002). Optical probe (8004) is provided as an NIR source located within the catheter probe.

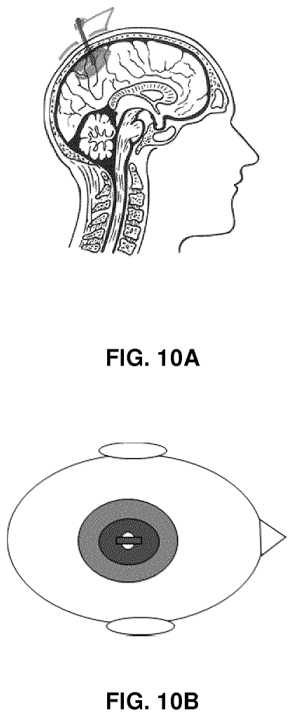

FIGS. 10A and 10B describe an alternative embodiment wherein the catheter accesses the site of the injury in the intracranial region through an opening in the middle of the imaging plate.

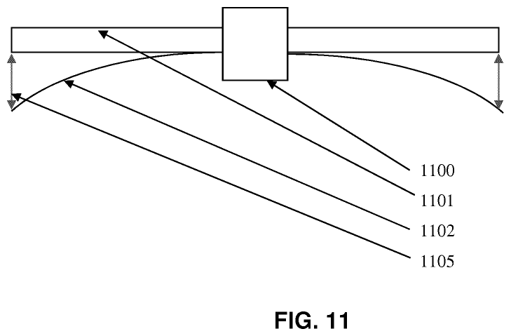

FIG. 11 describes an embodiment comprising a fixed plate (1101) in combination with a flexible imaging plate (1102) that conforms to the surface of the patient's head. The fixed plate (1101) comprises a ferule (1100) located in fixed association with the patient's head, allowing the underlying flexible plate (1102) to be deformed to conform to the patient's head. The flexible plate (1102) further comprises N (e.g. 4) displacement sensors (1105) on it to measure the degree of deformation. The number of displacement sensors (N) will determine image accuracy. The catheter accesses the site of the injury through the ferule (1100).

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

1. A system for intracranial imaging and treatment of an intracranial region, the system comprising:
(a) a catheter probe suitable for insertion into the intracranial region, the catheter probe comprising:
(i) a catheter housing; and
(ii) an optical probe comprising one or more optical emitters, wherein the optical probe is located within the housing;
(b) an imaging plate configured for fixed attachment through a plurality of attachment points to a surface of the intracranial region being imaged and treated, the imaging plate comprising an array of sensors, each sensor comprising an optical receiver;
wherein the one or more optical emitters is configured to emit light in proximity to the region being imaged, and the array of sensors is configured to measure transmitted light to determine the status of the region being imaged.

2. The system of claim 1, further comprising a surgical tool located within the housing.

3. The system of claim 2, wherein the region being imaged includes a bleed event, and the surgical tool is configured to treat the bleed event.

4. The system of claim 3, wherein the surgical tool is a syringe.

5. The system of claim 2, wherein the surgical tool is a syringe, a cauterization probe, an irrigation jet, or an electrode stimulator.

6. The system of claim 1, further comprising:
(c) one or more mechanical linkages connecting the catheter probe and the imaging plate; wherein the mechanical linkage is a mechanical arm configured to control the position of the catheter probe relative to the imaging plate.

7. The system of claim 1, wherein the fixed attachment of the imaging plate is achieved by screw attachment, adhesive attachment or other biologically suitable attachment means.

8. The system of claim 1, wherein the catheter probe further comprises a position control mechanism for controlling the position of the optical probe relative to the surgical tool within the catheter housing.

9. The system of claim 1, wherein each sensor further comprises a fixed housing, a plunger located within the fixed housing, the plunger being associated with the optical receiver, a positional sensor configured to measure radial displacement of the sensor, and a biasing mechanism configured to bias the receiver in contact with the surface of the intracranial region being imaged.

10. The system of claim 1, wherein the optical emitter is an LED.

11. The system of claim 10, wherein the LED emits near infrared light.

12. The system of claim 1, wherein the optical emitter is a laser diode.

13. The system of claim 1, wherein the imaging plate is flexible to conform to the surface of the intracranial region being imaged.

14. The system of claim 1, wherein the imaging plate comprises an opening located in the middle of the plate to provide access to region being imaged.

15. A method for imaging and treatment of an intracranial region of a subject, comprising the steps of:
providing a system comprising:
(a) a catheter probe suitable for insertion into the intracranial region, the catheter probe comprising:
(i) a catheter housing; and
(ii) an optical probe comprising one or more optical emitters, wherein the optical probe is located within the housing;
(b) an imaging plate configured for fixed attachment through a plurality of attachment points to a surface of the intracranial region being imaged and treated, the imaging plate comprising an array of sensors, each sensor comprising an optical receiver;
wherein the one or more optical emitters is configured to emit light in proximity to the region being imaged, and the array of sensors is configured to measure transmitted light to determine the status of the region being imaged;
attaching the imaging plate through a plurality of attachment points to a surface of the intracranial region of the subject;
inserting the catheter probe into the intracranial region of the subject;
obtaining an image of the intracranial region by interrogating the intracranial region with light emitted by the one or more optical emitters and detecting transmitted light with the sensors located on the imaging plate.

16. The method of claim 15, wherein the system further comprises a surgical tool located within the housing, and the method further comprises the step of deploying the surgical tool to treat the intracranial region.

17. The method of claim 16, wherein the intracranial region being imaged includes a bleed event, and the surgical tool is configured to treat the bleed event.

18. The method of claim 17, wherein the surgical tool is a syringe.

19. The method of claim 16, wherein the surgical tool is a syringe, a cauterization probe, an irrigation jet, or an electrode stimulator.

20. The method of claim 15, wherein each sensor further comprises a fixed housing, a plunger located within the fixed housing, the plunger being associated with the optical receiver, a positional sensor configured to measure radial displacement of the sensor, and a biasing mechanism configured to bias the receiver in contact with the surface of the intracranial region being imaged.

21. The method of claim 15, wherein the optical emitter is an LED.

22. The method of claim 21, wherein the LED emits near infrared light.

23. The method of claim 15, wherein the optical emitter is a laser diode.

24. The method of claim 15, wherein the imaging plate is flexible to conform to the surface of the intracranial region being imaged.

25. The method of claim 15, wherein the imaging plate comprises an opening located in the middle of the plate to provide access to the region being imaged.

26. The method of claim 15, wherein the fixed attachment of the imaging plate is achieved by screw attachment, adhesive attachment or other biologically suitable attachment means.

27. A system for intracranial imaging and treatment of an intracranial region, the system comprising:
(a) an imaging subsystem comprising
(i) an optical probe comprising one or more optical emitters located within a catheter housing; and
(ii) an imaging plate configured for fixed attachment through a plurality of attachment points to a surface of the intracranial region being imaged and treated, the imaging plate comprising an array of sensors, each sensor comprising an optical receiver; and
(b) an optional treatment subsystem comprising:
(i) a surgical tool located within the catheter housing,
wherein the one or more optical emitters is configured to emit light in proximity to the region being imaged, and the array of sensors is configured to measure transmitted light to determine the status of the region being imaged.

28-39. (canceled)
